(19) United States
(12) Patent Application Publication
(10) Pub. No.: US 2025/0198954 A1
(43) Pub. Date: Jun. 19, 2025

(54) METHOD FOR DIAGNOSING THYROID CARCINOMA

(71) Applicants: B. G. NEGEV TECHNOLOGIES AND APPLICATIONS LTD., AT BEN-GURION UNIVERSITY, Beer-Sheva (IL); MOR RESEARCH APPLICATIONS LTD., Ramat Gan (IL)

(72) Inventors: Lotem GOTNAYER, Beer-Sheva (IL); Netta VIDAVSKY, Beer-Sheva (IL); Merav FRAENKEL, Nof Ayalon (IL); Uri YOEL, Beer-Sheva (IL)

(73) Assignees: B. G. NEGEV TECHNOLOGIES AND APPLICATIONS LTD., AT BEN-GURION UNIVERSITY, Beer-Sheva (IL); MOR RESEARCH APPLICATIONS LTD., Ramat Gan (IL)

(21) Appl. No.: 18/851,671

(22) PCT Filed: Mar. 29, 2023
(86) PCT No.: PCT/IL2023/050330

§ 371 Date: Sep. 26, 2024

Related U.S. Application Data
(60) Provisional application No. 63/324,639, filed on Mar. 29, 2022.
(60) Provisional application No. 63/412,460, filed on Oct. 2, 2022.

(51) Int. Cl. G01N 23/22; A61B 10/02; G01N 33/483

(52) U.S. Cl. CPC G01N 23/22; A61B 10/0283; G01N 33/4833; G01N 2800/7028

(57) ABSTRACT

The present invention is directed to a method for diagnosing a thyroid cancer in a subject in need thereof, the method including determining presence, amount, or both, of at least one species of Zinc, Magnesium or both, wherein the species is in a microscopic calcification (MC) derived from a thyroid biopsy obtained from the subject, wherein: (a) the presence of the species; (b) alteration in the amount of the species compared to a healthy control; or (c) both, in the MC is indicative of the subject being afflicted with the thyroid cancer. Further provided are a method for treating a subject diagnosed with thyroid cancer, as well as preparing a sample including a thyroid biopsy for thyroid cancer diagnosis.

### CROSS-REFERENCE TO RELATED APPLICATIONS

This application claims the benefit of priority of U.S. Provisional Patent Application Nos. 63/324,639 filed Mar. 29, 2022, and 63/412,460 filed Oct. 2, 2022, both titled “FINE NEEDLE ASPIRATION CALCIFICATIONS AS A MARKER OF THYROID NODULE MALIGNANCY”. The contents of which are all incorporate herein by reference in their entirety.

### FIELD OF THE INVENTION

The present invention relates to, inter alia, thyroid cancer, diagnosis, treatment, or both.

### BACKGROUND OF THE INVENTION

Thyroid nodules (TN) are a common clinical problem, as they are present in over 60% of the population as detected via ultrasonography (US). Most TNs are benign and can be safely managed through routine monitoring, yet 5-10% are cancerous. Neck US is the first-line tool used to estimate the risk for malignancy (ROM) of a given TN. When US characteristics are consistent with an intermediate or high suspicion for TN malignancy, fine needle aspiration for cytology (FNAC) is indicated.

The main limitation of FNAC is that cytological results can be indeterminate in up to 30% of the cases, necessitating reevaluation through repeated FNAC, expensive molecular testing, or diagnostic thyroid lobe resection. As such, there is still a great need for improved diagnostic algorithms for TNs without subjecting patients to additional invasive procedures.

### SUMMARY OF THE INVENTION

The present invention, in some embodiments, is based, at least in part, on the surprising findings of a correlation between elemental composition, morphology, crystal phases, or any combination thereof, of microscopic calcification(s) (MC) derived from a thyroid biopsy and thyroid malignancy. A significant difference between the levels of Zinc, Magnesium, or both, in MC isolated from benign and malignant thyroid nodule (TN) was observed, thus, demonstrating that the presence/absence of Zinc deposit(s), Magnesium deposit(s), or both, within MC extracted from a thyroid biopsy that is being collected during routine fine needle aspiration for cytology (FNAC), can act as a biomarker for TN malignancy.

According to one aspect, there is provided a method for diagnosing a thyroid cancer in a subject in need thereof, the method comprising determining presence, amount, or both, of at least one species of Zinc, Magnesium or both, wherein the species is in a microscopic calcification (MC) derived from a thyroid biopsy obtained from the subject, wherein: (a) the presence of the species; (b) alteration in the amount of the species compared to a healthy control; or (c) both, in the MC is indicative of the subject being afflicted with the thyroid cancer, thereby diagnosing thyroid cancer in the subject.

According to another aspect, there is provided a method for diagnosing and treating a thyroid cancer in a subject in need thereof, the method comprising: (i) determining presence, amount, or both, of at least one species of Zinc, Magnesium, or both, wherein the species is in a MC derived from a thyroid biopsy obtained from the subject, wherein: (a) the presence of the species; (b) alteration in the amount of the species compared to a healthy control; or (c) both, in the MC is indicative of the subject being afflicted with the thyroid cancer, thereby diagnosing thyroid cancer in the subject; and (ii) administering to the subject diagnosed with thyroid cancer of step (a) a therapeutically effective amount of an anti-thyroid cancer therapy.

According to another aspect, there is provided a method for treating thyroid cancer in a subject diagnosed therewith, the method comprising administering to the subject a therapeutically effective amount of an anti-thyroid cancer therapy, wherein the diagnosed is by determination of: (a) presence of at least one species selected from: Zn, Mg, or both; (b) alteration of amount of the species compared to a healthy control; or (c) both (a) and (b), in a sample comprising a thyroid biopsy being obtained or derived from the subject, wherein said species is in a MC.

According to another aspect, there is provided a method for preparing a sample comprising a thyroid biopsy for thyroid cancer diagnosis, the method comprising providing a sample comprising a thyroid biopsy and extracting a MC comprising at least one species selected from: Zn, Mg, or both, from the thyroid biopsy, thereby preparing the sample comprising a thyroid biopsy for cancer diagnosis.

In some embodiments, the at least one species comprises Zinc, and presence of the Zinc in the MC is indicative of the presence of at least one malignant thyroid nodule in the thyroid biopsy.

In some embodiments, the absence of the Zinc in the MC is indicative of the presence of a benign thyroid nodule in the thyroid biopsy, absence of a malignant thyroid nodule in the thyroid biopsy, or both.

In some embodiments, thyroid cancer is differentiated thyroid carcinoma.

In some embodiments, the differentiated thyroid carcinoma is papillary thyroid carcinoma.

In some embodiments, the healthy control comprises at least one benign thyroid nodule and is devoid of a malignant thyroid nodule.

In some embodiments, the method further comprises a step before the determining, comprising obtaining a thyroid biopsy from the subject.

In some embodiments, obtaining is by fine-needle aspiration (FNA). In some embodiments, the thyroid biopsy is obtained by FNA.

In some embodiments, the method further comprises a step after the obtaining and before the determining, comprising preparing the sample for X-ray based analysis.

In some embodiments, the X-ray based analysis comprises energy dispersive X-ray spectroscopy (EDS).

In some embodiments, the method further comprises a step after the obtaining and before the determining, comprising preparing the sample for inductively coupled plasma (ICP).

In some embodiments, a determined wt % percentage of at least 0.09 wt % of said Zinc in said MC is indicative of the presence of at least one malignant thyroid nodule in said thyroid biopsy.

In some embodiments, determined is by ICP.

In some embodiments, extracting comprises subjecting the sample to a fixation solution, eliminating or removing an organic material from the fixed sample, or both.

In some embodiments, eliminating or removing the organic material comprises subjecting the fixed sample to a basic solution.

In some embodiments, the basic solution comprises sodium hypochlorite.

In some embodiments, the prepared sample is suitable for determination of presence, amount, or both, of the at least one species selected from: Zn, Mg, or both, and being in a MC, by an X-ray based methodology.

In some embodiments, the X-ray based methodology comprises EDS.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 includes a flow chart of a non-limiting study rationale.

FIG. 2 includes a flow chart of the current study.

FIGS. 3A-3D include images and a sonogram. (3A) Ultrasound (US) sonogram of a suspicious thyroid nodule (TN) during a US-guided fine needle aspiration (FNA) procedure. The FNA needle during nodule sampling is shown (arrow). Suspicious punctate calcifications were observed (including some marked with circles). (3B) The residual material left after FNA cytological evaluation following fixation with ethanol. (3C) After washing and centrifugation, the reddish FNA material contains organic and inorganic material. (3D) The inorganic precipitate remained after sodium hypochlorite treatment. This inorganic material was evaluated for MCs via a chemical analysis approach.

FIGS. 4A-4B include Fourier-transform infrared (FTIR) spectra of FNA MC from patients with benign TN (4A) and patients with a final diagnosis of thyroid carcinoma (4B). Each spectrum represents a different patient.

FIGS. 5A-5D include micrographs and spectra showing the morphology and elemental composition of MC derived from benign and malignant TN. (5A) and (5C) include scanning electron microscopy (SEM) and energy-dispersive X-ray spectroscopy (EDS) characterization of benign MC, respectively. (5B) and (5D) include SEM and EDS characterization of malignant MC, respectively.

### DETAILED DESCRIPTION

According to one aspect, there is provided a method for diagnosing a thyroid cancer in a subject in need thereof, the method comprising determining presence, amount, or both, of at least one species of Zinc (Zn), Magnesium (Mg) or both, in a thyroid biopsy obtained or derived from the subject. In some embodiments, the species is in a microscopic calcification (MC) derived from a thyroid biopsy obtained from a subject. In some embodiments, (a) the presence of the species; (b) alteration in the amount of the species compared to a control; or (c) both, in the MC is indicative of the subject being afflicted with thyroid cancer. In some embodiments, (a) the presence of the species; (b) alteration in the amount of the species compared to a control; or (c) both, in the MC is indicative of the subject comprising at least one malignant thyroid nodule (TN).

In some embodiments, (a) the absence of the species; (b) an amount of the species being essentially similar to a control; or (c) both, in the MC is indicative of the subject not being afflicted with thyroid cancer. In some embodiments, (a) the absence of the species; (b) an amount of the species being essentially similar to a control; or (c) both, in the MC is indicative of the subject comprising only benign TN, being devoid of malignant TN, or both.

According to another aspect, there is provided a method for diagnosing and treating a thyroid cancer in a subject in need thereof, the method comprising: (i) determining presence, amount, or both, of at least one species of Zn, Mg or both, in a thyroid biopsy obtained or derived from a subject, wherein: (a) the presence of the species; (b) alteration in the amount of the species compared to a control; or (c) both, in thyroid biopsy is indicative of the subject being afflicted with a thyroid cancer, thereby diagnosing thyroid cancer in the subject; and (ii) administering to the subject diagnosed with thyroid cancer of step (a) a therapeutically effective amount of an anti-thyroid cancer therapy.

As used herein, the terms “microscopic calcification”, “MC”, “mineral deposit”, and “solid deposit” are interchangeable, and refer to one or more or at least one species selected from: Calcium, Phosphorus, Iron, Aluminum, Chloride, Sodium, Potassium, Zinc, Magnesium, or any combination thereof being in a solid state, and insoluble.

In some embodiments, MC is insoluble. In some embodiments, insolubility is in an aqueous solution or water. Non-limiting examples for aqueous solution include, but are not limited to, a buffer (e.g., phosphate buffer, bicarbonate buffer, etc.), a base solution (e.g., bicarbonate, sodium hydroxide, etc.), or a salt solution (e.g., brine, saline, etc.). In some embodiments, MC is insoluble in a basic solution. In some embodiments, MC is insoluble in a hypochlorite containing solution. In some embodiments, a hypochlorite containing solution comprises any one of sodium hypochlorite and potassium hypochlorite. In some embodiments, insolubility is in a pH between 6 and 8, between 6 and 6.5, between 6 and 7, between 6 and 7.5, between 6.5 and 7.5, between 6.8 and 7.2. Each possibility represents a separate embodiment of the invention. In some embodiments, insolubility is in at a temperature between 20 and 40° C., between 2° and 25° C., between 2° and 30° C. between 2° and 35° C., between 25 and 35° C., between 3° and 40° C., or between 35 and 38° C. Each possibility represents a separate embodiment of the invention. In some embodiments, MC is insoluble in an aqueous solution, at a pH ranging between 6 and 8, and a temperature ranging between 2° and 40° C.

A used herein, the term “insoluble” refers to a solubility of at most 10 ppm, at most 5 ppm, at most 1 ppm, in an aqueous solution. In some embodiments, MC is soluble in an acidic solution, such as, but not limited to nitric acid.

In some embodiments, MC is in a solid state. In some embodiments, solid state comprises: (i) an amorphous state, (ii) a crystalline state, or (iii) any combination thereof. In some embodiments, solid state comprises an amorphous state. In some embodiments, at least 50%, least 60%, at least 70% of the species in a MC are in a non-crystalline state, or any value and range therebetween. In some embodiments, between 50 and 100%, between 50 and 70%, between 50 and 80%, between 50 and 90%, between 60 and 90%, between 60 and 100%, between 70 and 100%, or between 70 and 90%, of the species in a MC are in a non-crystalline state. Each possibility represents a separate embodiment of the invention.

In some embodiments, solid state comprises a crystalline state. In some embodiments, at least 50%, least 60%, at least 70% of the species in a MC are in a crystalline state, or any value and range therebetween. In some embodiments, between 50 and 100%, between 50 and 70%, between 50 and 80%, between 50 and 90%, between 60 and 90%, between 60 and 100%, between 70 and 100%, or between 70 and 90%, of the species in a MC are in a crystalline state. Each possibility represents a separate embodiment of the invention.

In some embodiments, MC consists essentially of calcium and phosphorous. In some embodiments, consists or consisting essentially of comprises at least 80%, at least 85%, at least 90%, at least 95%, at least 97%, or any value and range therebetween, of calcium phosphate. Each possibility represents a separate embodiment of the invention. In some embodiments, MC comprises between 80 and 100%, between 80 and 90%, between 80 and 95%, between 85 and 95%, or between 90 and 100%, of calcium phosphate. Each possibility represents a separate embodiment of the invention. Methods and means for determining crystallinity and/or amorphousness of an element(s), a solid(s), an insoluble sediment(s), and the like, such as disclosed herein, are known to a skilled in the art, such as X-Ray based methods, inclusive of X-ray diffraction.

In some embodiments, thyroid cancer comprises differentiated thyroid carcinoma. In some embodiments, differentiated thyroid carcinoma is selected from: papillary carcinoma, follicular carcinoma, Hurthle carcinoma, or a combination thereof.

In some embodiments, the presence of Zn in a MC is indicative of the presence of at least one malignant thyroid nodule in a thyroid biopsy. In some embodiments, absence of Zn in a MC is indicative of the presence of at least one benign thyroid nodule in a thyroid biopsy, the absence of a malignant thyroid nodule in a thyroid biopsy, or both. In some embodiments, the absence of Zn in the MC is indicative of the presence of at least one benign thyroid nodule in the thyroid biopsy. In some embodiments, the absence of Zn in a MC is indicative of absence of a malignant thyroid nodule in the thyroid biopsy.

In some embodiments, an increase of at least 55%, at least 65%, at least 75%, at least 85%, at least 90%, at least 95%, or any value and range therebetween, in the amount of Mg in a MC derived from a thyroid biopsy obtained from a subject compared to a control, is indicative of the presence of at least one malignant thyroid nodule in a thyroid biopsy.

In some embodiments, an increase of at least 55%, at least 65%, at least 75%, at least 85%, at least 90%, at least 95%, or any value and range therebetween, in the amount of Mg in a MC derived from a thyroid biopsy obtained from a subject compared to a control, is indicative of the subject being afflicted with thyroid cancer. Each possibility represents a separate embodiment of the invention.

In some embodiments, an increase of between 65 and 300%, between 65 and 200%, between 100 and 300%, between 100 and 200%, between 150 and 250%, in the amount of Mg in a MC derived from a thyroid biopsy obtained from a subject compared to a control, is indicative of: the presence of at least one malignant thyroid nodule in a thyroid biopsy, the subject being afflicted with thyroid cancer, or both. Each possibility represents a separate embodiment of the invention.

In some embodiments, the at least one species comprises at least two species. In some embodiments, the at least two species comprise: Zn or Mg, and one or more species selected from: Calcium (Ca), Phosphorus (P), Iron (Fe), Aluminum (Al), Chloride (Cl), Sodium (Na), Potassium (K), or any combination thereof.

In some embodiments, at least two species comprise: Zn and any one of the species: Ca, P, Fe, Al, Cl, Na, K or any combination thereof.

In some embodiments, at least two species comprise: Mg and any one of the species: Ca, P, Fe, Al, Cl, Na, K or any combination thereof.

In some embodiments, at least two species comprise: Zn and Mg.

In some embodiments, alteration means an increase or a decrease in the amount of any one of or one or more of the species: Ca, P, Fe, Al, CI, Na, K, or any combination thereof, in a MC derived from a thyroid biopsy obtained from a subject. In some embodiments, increase or decrease is compared to the control. In some embodiments, an alteration in any one of the species: Ca, P, Fe, Al, Cl, Na, K, in a MC derived from a thyroid biopsy obtained from a subject compared to the control, is indicative of the subject being afflicted with thyroid cancer. In some embodiments, any one of: (i) a decrease of at least 10% in the amount of the K in the MC derived from the thyroid biopsy compared to the control; (ii) an increase of at least 20% in the amount of the Ca in the MC derived from the thyroid biopsy compared to the control; (iii) a decrease of at least 10% in the amount of the Fe in the MC derived from the thyroid biopsy compared to the control; (iv) an increase of at least 10% in the amount of the P in the MC derived from the thyroid biopsy compared to the control; (v) an increase of at least 10% in the amount of the Cl in the MC derived from the thyroid biopsy compared to the control; (vi) an increase of at least 25% in the amount of the Na in the MC derived from the thyroid biopsy compared to the control; and (vii) any combination of (i) to (vi), is indicative of the subject being afflicted with thyroid cancer.

In some embodiments, the control comprises or is derived from a healthy thyroid tissue. In some embodiments, a healthy thyroid tissue is obtained from a healthy subject. In some embodiments, a healthy thyroid tissue is obtained from the subject afflicted with cancer before being afflicted with cancer. In some embodiments, a healthy thyroid tissue is obtained from a subject afflicted with cancer. In some embodiments, the control comprises a MC derived from at least one benign thyroid nodule. In some embodiments, the control comprises a MC, derived from a thyroid nodule being devoid of malignant cells. In some embodiments, the control is devoid of a malignant thyroid nodule, and/or cells.

In some embodiments, an increase in the amount of a specie (such as Zn) in a MC derived from the thyroid biopsy is an increase of at most 2,000%, at most 1,500%, at most 1,000%, compared to a control, or any value and range therebetween. Each possibility represents a separate embodiment of the invention.

In some embodiments, an increase in the amount of a specie (such as Zn) in a MC derived from the thyroid biopsy is an increase of between 10 and 2,000%, between 10 and 1,500%, between 10 and 1,000%, between 20 and 500%, compared to a control. Each possibility represents a separate embodiment of the invention.

In some embodiments, a decrease in the amount of a specie (such as K) in a MC derived from the thyroid biopsy is a decrease of at least 70%, at least 75%, at least 80%, at least 90%, or any value and range therebetween, compared to a control. Each possibility represents a separate embodiment of the invention.

In some embodiments, a decrease in the amount of a specie (such as K) in a MC derived from the thyroid biopsy is a decrease of between, 70 and 100%, between 70 and 90%, between 70 and 80%, between 75 and 85%, between 75 and 95%, between 80 and 100%, between 85 and 95%, between 90 and 100%, compared to a control. Each possibility represents a separate embodiment of the invention.

In some embodiments, a decrease in the amount of K in a MC derived from the thyroid biopsy is a decrease of at least 10%, at least 15%, at least 20%, or any value and range therebetween, compared to a control. Each possibility represents a separate embodiment of the invention.

In some embodiments, a decrease in the amount of K in a MC derived from the thyroid biopsy is a decrease of between 10% and 100%, between 10 and 75%, between 15 and 50%, between 10 and 30%, between 15 and 40%, between 10 and 50%, or between 20 and 100%, compared to a control. Each possibility represents a separate embodiment of the invention.

In some embodiments, an increase in the amount of Ca in a MC derived from the thyroid biopsy is an increase of at least 20%, at least 25%, at least 30%, or any value and range therebetween, compared to a control. Each possibility represents a separate embodiment of the invention.

In some embodiments, an increase in the amount of Ca in a MC derived from the thyroid biopsy is an increase of between 20% and 100%, between 20 and 80%, between 20 and 60%, between 20 and 40%, between 25 and 40%, or between 30 and 40%, compared to a control. Each possibility represents a separate embodiment of the invention.

In some embodiments, a decrease in the amount Fe in a MC derived from the thyroid biopsy is a decrease of at least 10%, at least 15%, or any value and range therebetween, compared to a control. Each possibility represents a separate embodiment of the invention.

In some embodiments, a decrease in the amount of Fe in a MC derived from the thyroid biopsy is a decrease of between 10 and 100%, between 10 and 50%, between 10 and 20%, between 15 and 90%, between 10 and 30%, between 10 and 25%, or between 50 and 100%, compared to a control. Each possibility represents a separate embodiment of the invention.

In some embodiments, an increase in the amount P in a MC derived from the thyroid biopsy is an increase of at least 10%, at least 15%, at least 20%, or any value and range therebetween, compared to a control. Each possibility represents a separate embodiment of the invention.

In some embodiments, an increase in the amount of P in a MC derived from the thyroid biopsy is an increase of between 10 and 100%, between 10 30%, between 10 and 20%, between 15 and 50%, between 10 and 15, between 10 and 50%, or between 50 and 100%, compared to a control. Each possibility represents a separate embodiment of the invention.

In some embodiments, an increase in the amount of Cl in a MC derived from the thyroid biopsy is an increase of at least 10%, at least 15%, at least 20%, or any value and range therebetween, compared to a control. Each possibility represents a separate embodiment of the invention.

In some embodiments, an increase in the amount of Cl in a MC derived from the thyroid biopsy is an increase of between 10% and 100%, between 10 and 80%, between 10 and 50%, between 10 and 30%, between 50 and 80%, between 70 and 90%, or between 10 and 40%, compared to a control. Each possibility represents a separate embodiment of the invention.

In some embodiments, an increase in the amount of Na in a MC derived from the thyroid biopsy is an increase of at least 25%, at least 35%, at least 40%, or any value and range therebetween, compared to a control. Each possibility represents a separate embodiment of the invention.

In some embodiments, an increase in the amount of Na in a MC derived from the thyroid biopsy is an increase of between 25 and 200%, between 25 and 175%, between 40 and 60%, between 35 and 75%, between 50 and 150%, between 100 and 200, between 50 and 90%, between 65 and 85%, or between 50 and 100%, compared to a control. Each possibility represents a separate embodiment of the invention.

In some embodiments, determining presence, amount, or both, of any one of the species: Ca, P, Fe, Al, Cl, Na, K, Zn, Mg is by X-ray based methodology. In some embodiments, determining presence of any one of the species: Ca, P, Fe, Al, Cl, Na, K, Zn, Mg is by X-ray based methodology. In some embodiments, determining amount of any one of the species: Ca, P, Fe, Al, Cl, Na, K, Zn, Mg is by X-ray based methodology. In some embodiments, X-ray based methodology comprises energy dispersive X-ray spectroscopy (EDS).

In some embodiments, determining presence, amount, or both, of any one of the species: Ca, P, Fe, Al, Cl, Na, K, Zn, Mg is by inductively coupled plasma (ICP). In some embodiments, determining presence of any one of the species: Ca, P, Fe, Al, Cl, Na, K, Zn, Mg is by ICP. In some embodiments, determining amount of any one of the species: Ca, P, Fe, Al, Cl, Na, K, Zn, Mg is by ICP.

In some embodiments, benign TN is characterized by a weight percentage of Zn within the MC of at most 0.08 wt %, at most 0.075 wt %, or at most 0.07 wt %, as determined by ICP. Each possibility represents a separate embodiment of the invention. In some embodiments, benign TN is characterized by a weight percentage of Zn within the MC ranging between 0 and 0.08 wt %, between, 0 and 0.07 wt %, 0 and 0.06 wt %, between 0 and 0.05 wt %, between 0 and 0.04 wt %, between 0 and 0.03 wt %, between 0 and 0.02 wt %, between 0 and 0.01 wt %, or between 0.02 and 0.07 as determined by ICP. Each possibility represents a separate embodiment of the invention.

In some embodiments, malignant TN is characterized by a weight percentage of Zn within the MC of at least 0.09 wt %, at least 0.1 wt %, or at least 0.2 wt %, or any range or value in between, as determined by ICP. Each possibility represents a separate embodiment of the invention. In some embodiments, malignant TNs are characterized by an amount Zn within the MC ranging between 0.09 and 5 wt %, between, 0.1 and 0.5 wt %, 0.1 and 1 wt % between 0.2 and 1 wt %, between 0.2 and 0.8 wt %, between 0.2 and 2 wt %, or between 0.3 and 10 wt %, as determined by ICP. Each possibility represents a separate embodiment of the invention.

In some embodiments, the method further comprises a step before the determining, comprising obtaining a thyroid biopsy from the subject. In some embodiments, a thyroid biopsy is obtained by fine-needle aspiration (FNA). In some embodiments, a thyroid biopsy comprises a thyroid tissue of a subject. In some embodiments, a thyroid biopsy comprises thyroid tissue fragment and/or at least one thyroidal cell. In some embodiments, a tissue fragment is characterized by an average size distribution of at most 700 μm, at most 680 μm, 660 μm, or any value and range therebetween. Each possibility represents a separate embodiment of the invention. In some embodiments, the tissue fragments are characterized by an average size distribution of between 0.1 nm and 700 μm, between 0.1 and 0.5 μm, between 0.1 and 1 μm, between 0.1 and 10 μm, between 0.1 and 100 μm, between 0.1 and 200 μm, between 0.1 and 300 μm, between 0.1 and 400 μm, between 0.1 and 500 μm, between 0.1 and 600 μm, between 100 and 700 μm, or between 250 and 700 μm. Each possibility represents a separate embodiment of the invention. Methods for obtaining fragments of thyroid biopsy, as well as measuring their average size distribution are well known in the art. Non-limiting examples for such methods, include, but are not limited to, light scattering and dynamic light scattering.

In some embodiments the method further comprises determining the morphology, topography, or both, of a MC. In some embodiments, the method further comprises subjecting the MC to a surface morphology and or topography characterization method, thereby determining the morphology and or topography of the MC. In some embodiments, determining morphology comprises determining at least one parameter selected from: texture (e.g., smooth, rough, porous, granulated etc.), shape (sphere, granulates, irregular shape, etc.), size (sub-micron, micron, etc.), distribution (e.g., aggregates, particle density, etc.), or any combination thereof. In some embodiments, morphology characterization methods are well known to a skilled in the art, such as those described in Surface Treatment of Materials for Adhesive Bonding, Second Edition, Chapter 4, William Andrew publishing (2014) and Chem. Soc. Rev., 2020, 49, 3278-3296, which is incorporated herein by reference in its entirety.

In some embodiments, MC comprises a plurality of particles. As used herein, the term “plurality” encompasses any integer equal to or greater than 2. In some embodiments, a plurality comprises at least 2, at least 5, at least 10, at least 15, at least 20, or any value and range therebetween. Each possibility represents a separate embodiment of the invention.

In some embodiments, MC is derived from a benign TN. In some embodiments, a particle of a MC derived from a benign TN is characterized by an average size of at least 1 μm, at least 3 μm, at least 5 μm, or any value and range therebetween. Each possibility represents a separate embodiment of the invention. In some embodiments, a particle of a MC derived from a benign TN is characterized by an average size of between 1 and 20 μm, between 1 and 10 μm, between 1 and 5 μm, or between 5 and 10 μm. Each possibility represents a separate embodiment of the invention.

In some embodiments, MC is derived from a malignant TN. In some embodiments, a particle of a MC derived from a malignant TN is characterized by an average size of at most 1 μm, at most 0.8 μm, at most 0.5 μm, or any value and range therebetween. Each possibility represents a separate embodiment of the invention. In some embodiments, a particle of a MC derive from a malignant TN is characterized by an average size of between 100 and 1000 nm, between 100 and 500 nm, between 100 and 250 nm, between 500 and 1000 nm, between 100 and 750 nm, between 250 and 750 nm, between 250 and 500 nm, or between 500 and 750 nm. Each possibility represents a separate embodiment of the invention.

In some embodiments, the method further comprises a step after obtaining and before determining (hereinafter “sample preparation step”), comprising preparing the thyroid biopsy for elemental composition and morphology analysis (i.e., ICP, EDS, SEM). In some embodiments, the sample preparation step comprises: (i) fixing a thyroid biopsy in a fixation solution, (ii) centrifuging the fixed thyroid biopsy, (iii) eliminating, or removing organic material from the fixed thyroid biopsy, or (iv) any combination of (i) to (iii). In some embodiments, the sample preparation step comprises extracting a MC from a thyroid biopsy.

In some embodiments, the fixing step comprises contacting a thyroid biopsy with a fixation solution or a fixative. In some embodiments, the fixation solution comprises ethanol. In some embodiments, the fixation step is before a centrifugation step. In some embodiments, the sample preparation step further comprises an additional fixation step after centrifugation and before elimination or removing organic material. In some embodiments, a centrifugation step comprises centrifuging the fixed thyroid biopsy.

In some embodiments, eliminating or removing the organic material from the fixed thyroid biopsy comprises contacting the fixed thyroid biopsy with an effective amount of a basic solution, centrifuging the fixed thyroid biopsy contacted with the basic solution, or both.

As used here in the term “effective amount” refers to an amount of a basic solution applied to a sample, wherein the amount is sufficient to remove or eliminate at least 90%, at least 93%, at least 95%, at least 97%, of the organic material in the thyroid biopsy (e.g., a fixed thyroid biopsy), or any value and range therebetween. In some embodiments, at least 90%, at least 93%, or at least 95% of the organic material is removed or eliminated during the sample preparation step, or any value and range therebetween. Each possibly represents a separate embodiment of the invention. In some embodiments, between 90 and 100%, between 90 and 95%, between 93 and 100% of organic material are eliminated or removed during the sample preparation step. Each possibly represents a separate embodiment of the invention.

In some embodiments, a basic solution comprises or is hypochlorite-based solution. In some embodiments, the hypochlorite solution comprises or is sodium hypochlorite.

In some embodiments, the method further comprises a washing step. In some embodiments, washing is by contacting the fixed thyroid biopsy to a solution (i.e., water or a solution comprising water), vortexing, inverting, shaking, centrifuging, or any combination thereof.

In some embodiments, the method further comprises administering to a subject diagnosed with thyroid cancer a therapeutically effective amount of an anti-thyroid cancer therapy. In some embodiments, anti-thyroid cancer therapy comprises iodine therapy, kinase inhibitors (e.g., Levatinib, Sorafenib, Cabozantinb, Vandetanib etc.), RET inhibitors (such as Selpercatinib and Pralsetinib), TRK inhibitors (Larotrectinib, entrectinib, etc.) or any combination thereof.

As used herein, the terms “administering,” refer to any method which, in sound medical practice, therapeutically effective amount of an anti-thyroid cancer therapy to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

As used herein the term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the subject is a mammal. In some embodiments, the mammal is a human. In some embodiments, a subject in need of diagnosis is at increased risk to developing thyroid cancer.

According to another aspect there is provided a method for treating thyroid cancer in a subject diagnosed therewith, the method comprising administering to the subject a therapeutically effective amount of an anti-thyroid cancer therapy.

In some embodiments, diagnosed is by determination of: (a) presence of at least one of species: Ca, P, Fe, Al, Cl, Na, K, Mg, Zn; (b) alteration of amount of the species compared to a control; or (c) both, in a sample comprising a thyroid biopsy being obtained or derived from the subject, wherein the species is in a MC.

According to another aspect there is provided a method for preparing a sample comprising a thyroid biopsy for thyroid cancer diagnosis, the method comprising providing a sample comprising a thyroid biopsy and extracting an MC comprising at least one of the species: Ca, P, Fe, Al, Cl, Na, K, Mg, Zn, thereby preparing the sample comprising a thyroid biopsy for cancer diagnosis.

In some embodiments, the thyroid biopsy comprises a thyroid tissue obtained or derived from a subject. In some embodiments, the thyroid biopsy comprises MC. In some embodiments, the thyroid biopsy is obtained by FNA.

In some embodiments, the extracting step comprises: (i) fixing the thyroid biopsy, (ii) eliminating, or removing organic material from the fixed thyroid biopsy, or (iii) both (i) and (ii). In some embodiments, step (i) and (ii) are sequential.

In some embodiments, fixing is by contacting a thyroid biopsy with a fixation solution. Fixation solutions and methods of using same would be apparent to a person of skill in the art of histology and pathology.

In some embodiments, the extracting step further comprises centrifuging the fixed thyroid biopsy before eliminating or removing the organic material.

In some embodiments, eliminating, or removing organic material from the fixed thyroid biopsy is by contacting the fixed thyroid biopsy with a basic solution, centrifuging, or both. In some embodiments, contacting comprising mixing the fixed thyroid biopsy with an effective amount of a basic solution. In some embodiments, the basic solution comprises or is sodium hydrochloride.

In some embodiments, at least 90%, at least 93%, or at least 95% of the organic material is removed or eliminated during the sample extraction step, or any value ad range therebetween. Each possibly represents a separate embodiment of the invention. In some embodiments, between 90 and 100%, between 90 and 95%, or between 93 and 100% of organic material are eliminated or removed during the extraction step. Each possibly represents a separate embodiment of the invention.

In some embodiments, the sample is suitable for determination of presence, amount, or both, of at least one species selected from: Ca, P, Fe, Al, Cl, Na, K, Zn, Mg, or any combination thereof. In some embodiments, the prepared sample is suitable for determination of presence of at least one species selected from: Ca, P, Fe, Al, Cl, Na, K, Zn, Mg, or any combination thereof. In some embodiments, the prepared sample is suitable for determination of amount of at least one species selected from: Ca, P, Fe, Al, Cl, Na, K, Zn, Mg, or any combination thereof. In some embodiments, determination of any one of the species selected from: Ca, P, Fe, Al, Cl, Na, K, Zn, Mg, or any combination thereof is by an X-ray spectroscopy methodology. In some embodiments, detection is by EDS. In some embodiments, detection is by ICP.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein the term “essentially similar” means that the amount of the species in the MC deviates by at most 40%, at most 30%, at most 10% from the amount of the species in a (MC) control. In some embodiments, the amount of the species in the MC are characterized by a deviation of 0 and 40%, between 0 and 10%, between 0 and 20%, between 0 and 30%, between 20 and 40%, from the control. Each possibility represents a separate embodiment of the invention.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Various embodiments and aspects of the present invention as delineated herein above and as claimed in the claims section below find experimental support in the following examples. Reference is now made to the following examples, which together with the above descriptions illustrate some embodiments of the invention in a non-limiting fashion.

### Materials and Methods

### Study Overview

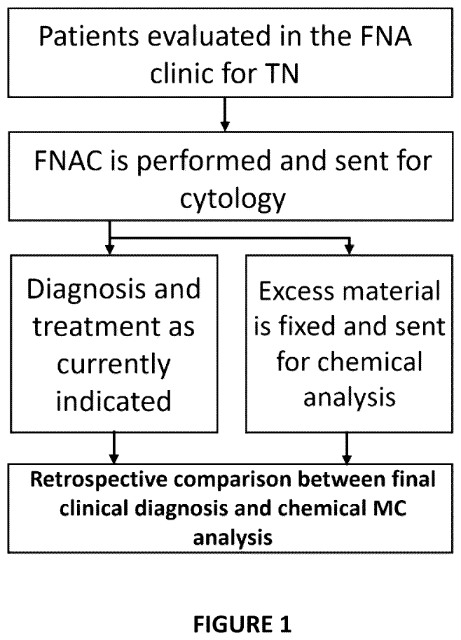

In this single-center, proof-of-concept, prospective study, the inventors assessed the diagnostic potential of MCs within TNs aspirated during routine FNAC procedures. The inventors mainly focused on the morphology and the elemental composition of MCs. Samples were collected between November 2020 and December 2022 during FNAC procedures conducted at the endocrine unit of Soroka University Medical Center (SUMC). Patients aged 18 years or older with TNs and an indication for FNAC, who agreed to sign the informed consent document were included in this study. Pregnant women were excluded. All patients with clinical diagnoses were included in the study cohort, and their data were deidentified and coded for analysis. Clinical decisions were made according to the current guidelines based on thyroid US and cytological findings. Comparisons between the final clinical diagnosis of a TN as benign or malignant and MC analysis results were conducted retrospectively (FIG. 1). The institutional review committee approved this study in advance of its execution (approval number 190-17-SOR, 27 Sep. 2017).

### US and US-Guided FNA, Cytological, and Molecular Evaluation

All recruited patients underwent US examination of the neck using a US scanner with a 12.5-MHz linear phased-array transducer. US-guided FNA of TNs was performed by an experienced endocrinologist, as clinically indicated according to the American College of Radiology TIRADS System. In general, TNs classified as TIRADS 5, 4, and 3 were biopsied when the largest TN diameter was greater than or equal to 1 cm, 1.5 cm, and 2.5 cm, respectively. FNAC was performed for TIRADS 1 and 2 TNs and for TIRADS 3 TNs<2.5 cm in diameter as clinically indicated (e.g., before thyroid lobectomy of the contralateral side). The US-guided FNA procedure was conducted using the “free-hand technique”, with a 22-G needle connected to a 3 mL syringe. After the needle was localized inside the TN, negative pressure was produced using the syringe handle while performing inward-outward and rotatory movements until bloody material was visible in the distal end of the syringe. The aspirate inside the needle was sent as a direct smear and a liquid-based biopsy (ThinPrep PreservCyt®, Hologic Ins.) for the formal cytological evaluation. The small amount of bloody material remaining in the syringe was fixed with 1 mL of 96% ethanol (Bio-Lab ltd) and sent for MC analysis. The cytological evaluation was conducted by experienced cytopathologists based on the 2017 Bethesda System for Reporting Thyroid Cytopathology. Molecular analysis of samples was offered to patients with indeterminate cytology (Bethesda categories III and IV).

### Clinical Diagnosis

TNs were diagnosed as TC based on a final histological report following partial or complete thyroidectomy. As minimally invasive follicular thyroid cancer (FTC) and non-invasive follicular thyroid neoplasms with papillary-like nuclear features (NIPTPs) are histological diagnoses, they were labeled as “neoplastic” lesions and for statistical analysis were considered as “malignant TN”. TNs were labeled as “benign” if they fulfilled one of the following criteria: (i) Benign US features (TIRADS categories 1-3) and a Bethesda category II cytology report, (ii) worrisome US features (TIRADS categories 4-5) with a Bethesda category II report in two separate FNAC analyses, (iii) benign US features and a Bethesda category III cytology report on initial FNAC analysis followed by a Bethesda category II cytology report upon follow-up FNAC, (iv) molecular testing performed for Bethesda category III or IV TNs with a ROM<5% irrespective of US characteristics, or (v) a final surgical pathological report indicating that a TN was benign.

MC Isolation from FNA Material

Two (2) mL of 70% ethanol (Bio-Lab ltd) were added to the fixed FNA material that was kept in the original syringe for overnight suspension, after which the samples were transferred to a 15 mL tube and centrifuged for 5 minutes at 4,000 rpm in a benchtop centrifuge (5810 R, Eppendorf International). This process was repeated three times, and then the samples were soaked in deionized water for two hours at 37° C. to decompose any remaining organic matter. Then, the FNA material was transferred to 2 mL tubes and centrifuged for 10 min at 20,000 rpm (5424 R, Eppendorf International). The supernatant was aspirated, and a solution of 1.2% w/w sodium hypochlorite (Liel POWER) in deionized water was added to the pellet overnight, followed by three rounds of washing with deionized water and centrifugation (20,000 rpm, 10 min). The supernatant was then aspirated, and absolute ethanol (Bio-Lab ltd) was added to the pellet for the storage of the remaining inorganic material.

### Imaging and Elemental Analysis of FNA MCs

The elemental composition of MCs was examined using a benchtop SEM Energy-dispersive X-ray spectroscopy (SEM-EDS) and validated the results via high-resolution SEM-EDS and ICP-OES (Inductively Coupled Plasma Optical Emission Spectroscopy). The rationale behind using a benchtop SEM-EDS strategy to characterize the composition of these MC samples was to explore the potential application of this methodological approach as a component of the routine pathological assessment of patients with suspicious TNs, which currently consists of US and cytological analyses.

For sizing and morphological characterization, FNA MCs were mounted on conductive carbon adhesive tape and analyzed with a field-emission SEM (Verois 460L, Thermo Fisher). All samples were measured with a benchtop SEM (Phenom, Thermo Fischer) equipped with EDS at 15 kV for elemental analysis. Backscattered and secondary electron imaging was carried out at 5 kV. Additionally, six samples were sputter-coated with carbon using a Quorum Q150T ES instrument and then measured using the Verios 460L EDS (Oxford Instruments) at 15 kV. ICP-OES-based validation of EDS results was performed for six benign and two malignant samples after EDS characterization.

FNA MCs were digested in 0.2 μL of ultrapure 70% HNO3 (Sigma-Aldrich) and diluted to a final volume of 5 mL prior to multi-elemental trace analyses performed using an ICP-OES instrument (Spectro Arcos) equipped with a CETAC autosampler. The instrument was calibrated with a multi-elemental standard solution dissolved in 5% HNO3 in the 0.01 to 20 ppm range. Operating conditions were as follows: 1400 W plasma RF power, 13 L/min coolant gas flow, 1 L/min auxiliary flow, 0.75 nebulizer flow, 30 rpm pump speed, and 15 s flush time between samples. All elements were detected in radial mode. For integration, 3 points per peak and 1 point for the background were used.

### FTIR Characterization of MCs

All isolated particles were analyzed via FTIR spectroscopy (Nicolet iS5, Thermo Fischer). Each sample was mixed with 200 mg of KBr (Sigma-Aldrich) to form a homogenous powder that was then pressed into a pellet for analysis. FTIR instrument was operated at a wavelength range between 450 and 4,000 cm-1 and 32 scans were performed at a resolution of 8 cm-1. Plotting and spectral analyses were performed with Origin 2020.

### Statistical Analysis

The results of MC chemical analyses with the final diagnostic results for included TNs were retrospectively compared. MCs were classified as either benign or TC and neoplastic TN, as detailed above. Clinical continuous variables are presented as medians and ranges, while categorical variables are presented as counts and percentages. Mann-Whitney tests were used to compare continuous variables (e.g., age, TN diameter), while categorical variables (e.g., elemental composition) were compared with chi-square tests.

A two-sided P<0.05 was considered statistically significant. For elemental composition, at least 5 individual MCs were measured by SEM-EDS for each patient and the concentration of each element in each patient is presented as the mean±standard deviation, with the amount of Zn detected per patient (w/w %) also being presented as the mean±standard deviation.

Power calculations were used to guide the design of this study, with initial calculations indicating that to achieve a sensitivity and specificity of 0.8 with a 95% confidence interval of ±0.10, 62 patients diagnosed with benign TNs, and 62 patients diagnosed with TC or neoplastic TN would be required. However, as unexpected MC chemical analysis results were observed based on data from just 52 patients, we present these data as a proof-of-concept for the ability of MCs to differentiate between benign and malignant TN.

### Example 1

### Clinical Data and the Development of an FNA MC Isolation Protocol

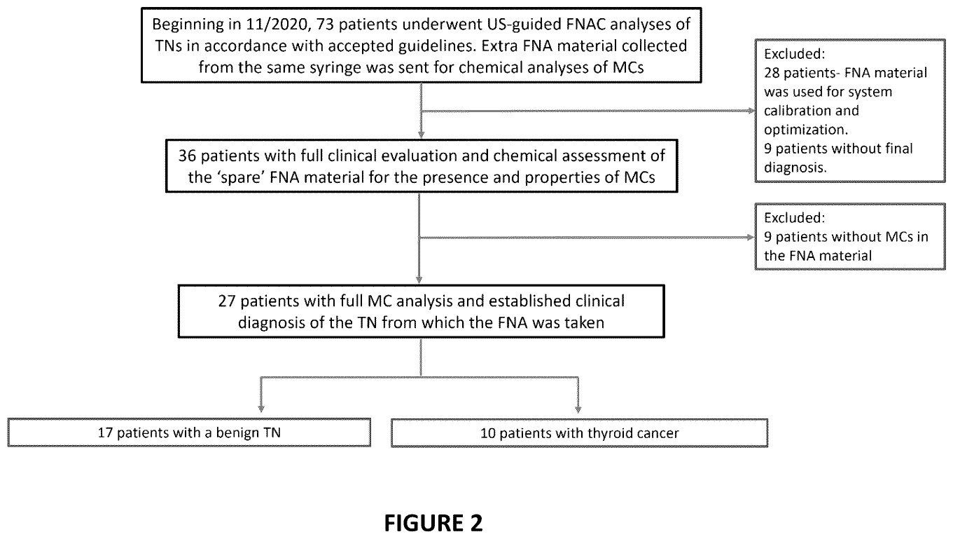

For this study, samples from 124 patients were collected during FNA procedures. Samples from 26 patients were used for protocol development, system calibration, and optimization, and 35 patients were still awaiting a final diagnosis. The remaining 63 patients underwent full clinical evaluation, and FNA samples from these patients were subject to the MC isolation protocol. In 52/63 patients (82.5%), FNA MCs were successfully identified using the established chemical analysis approach, while in 11/63 patients (17.5%), no MCs were identified. Interestingly, all 11 patients whose samples did not contain MCs were ultimately diagnosed with benign TNs. The final study cohort included 52 patients with complete clinical evaluation for whom MCs were identified in the FNA material (FIG. 2). Patients were divided according to their final diagnosis, including 30 patients with benign TNs (median age: 54 years [range: 26-76 years], 83.3% females), and 22 patients with TC or neoplastic TN (median age: 42 years [range: 18-80 years], 63.6% females). While any echogenic foci were observed by US in 32/52 patients (61.5%) of the entire cohort, punctate calcifications, which are the most suspicious echogenic foci, were reported in 10% and 77.3% of patients ultimately diagnosed with benign TN versus TC or neoplastic TN, respectively. In all cases where echogenic foci (of any type) were detected by US, MCs were observed in the FNA material following chemical analysis. Additionally, MCs were detected using chemical analysis in 13/30 patients (43%) who were ultimately diagnosed with benign TNs and in 3/22 patients (14%) with a final diagnosis of TC or neoplastic TN in whom echogenic foci were not detected by US (Table 1).

With respect to the results of the cytological evaluation, 23/30 (77%) of patients with a final diagnosis of benign TN had cytology results of Bethesda 2, while 7/30 patients (23%) had a cytological diagnosis of Bethesda 1 (2 patients) or Bethesda 3 (5 patients). Intriguingly, 11/22 (50%) of patients with a final diagnosis of TC or neoplastic TN were classified as having indeterminate cytology (Bethesda categories III or IV) following initial FNAC evaluation. Of 22 patients with a final diagnosis of TC or neoplastic TN, 17 patients had PTC, three had micro-PTC (sub-centimetric PTC), one had NIFTP, and one had minimally invasive FTC.

TABLE 1
Clinical and ultrasonographic characteristics of patients
with TNs grouped according to the final diagnosis

| | | | TC or | |
|---|---|---|---|---|
| | | Benign TN | neoplastic TN* | |
| | | N = 30 | N = 22 | P -value |
| Age (median, range) | | 54 (26-76) | 42 (18-80) | 0.18 |
| Female sex n (%) | | 25 (83.3) | 14 (63.6) | 0.11 |
| TN largest diameter (cm), | | 3.0 (1.2-9.0) | 2.2 (1.2-6.1) | 0.045 |
| median (range) | | | | |
| TN volume (mL), median | | 6.35 (0.42-72) | 3.35 (0.67-40) | 0.18 |
| (range) | | | | |
| TIRADS categories, median | | 3 (1-5) | 5 (3-5) | <0.001 |
| (range) | | | | |
| TIRADS points, median | | 3.5 (2-12) | 7.5 (3-13) | 0.027 |
| (range) | | | | |
| Echogenic | None | 17 (56.7) | 3 (13.6) | |
| foci | Comet tail | 2 (6.7) | 1 (4.5) | |
| per US | artifacts | | | |
| (n, %) | Macro- | 8 (26.7) | 0 | |
| | calcifications | | | |
| | Rim/peripheral | 0 | 1 (4.5) | |
| | calcifications | | | |
| | Punctate | 3 (10.0) | 17 (77.3) | |
| | calcifications | | | |

*Papillary thyroid cancer (PTC)- 17 patients, micro-PTC- 3 patients, Non-invasive follicular thyroid neoplasm with papillary-like nuclear features (NIFTP)- 1 patient, minimally invasive follicular thyroid cancer- 1 patient.
Abbreviations:
Thyroid Nodule (TN),
Ultrasound (US),
Thyroid Imaging Reporting and Data System (TIRADS),
Thyroid cancer (TC)

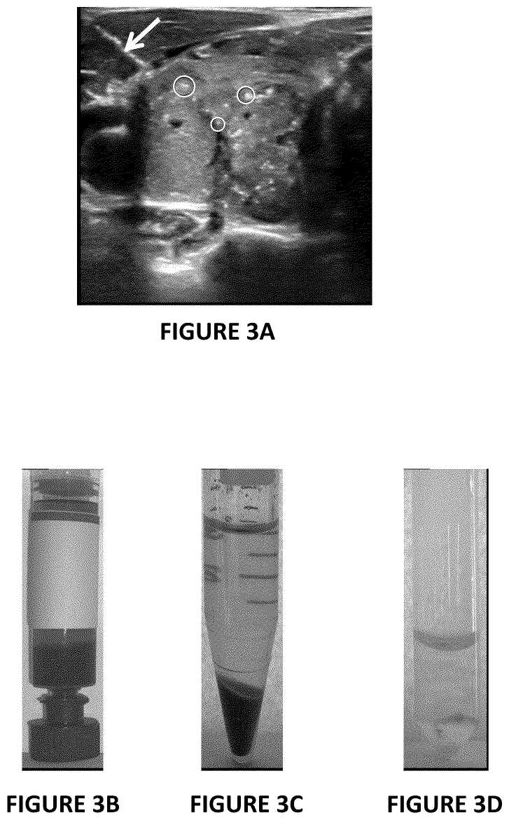

During the FNA procedure, some patients exhibited visible echogenic foci in the TN (FIG. 3A, Table 1). The remaining aspirated material was used for MC isolation following its fixation (FIG. 3B), washing (FIG. 3C), and incubation in a sodium hypochlorite solution (FIG. 3D), after which the inorganic precipitate underwent chemical analysis.

### Example 2

### The Crystal Phase of FNA MCs

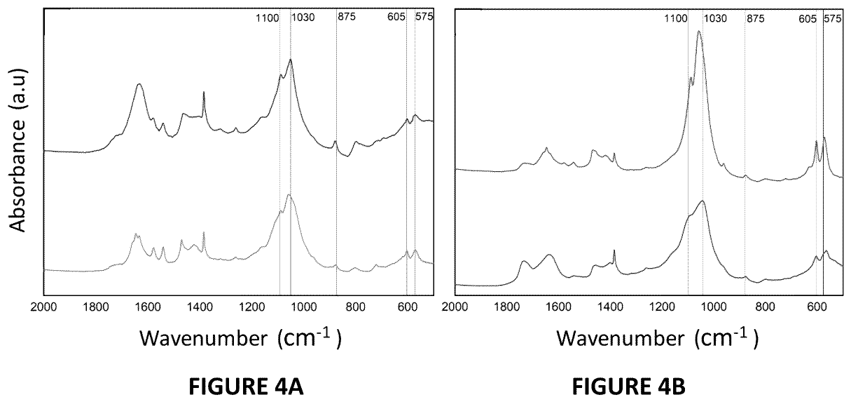

To determine whether MCs are present in the FNA material and to identify the crystal phases of these FNA MCs, the inventors used FTIR spectroscopy (FIG. 4). These MCs were found to contain phosphate and carbonate peaks (V3 PO4: 1,100 cm-1 and 1,030 cm-1, V4 PO4: 605 cm-1 and 575 cm-1, V2 CO3: 875 cm-1), consistent with a carbonated calcium phosphate apatite. The 1,386 cm-1 peak corresponds to nitrate, and some remaining organic material was also detected in these samples (1,600-1,800 cm-1). To ensure that the isolation process did not affect the spectral features of the MC crystals, the FTIR spectra of commercial hydroxyapatite crystals before and after the isolation process was compared, no significant difference was observed.

FTIR spectra revealed that the MCs were comprised of a mixture of crystal phases and that the spectral features of MCs isolated from patients in the same TN malignancy category were not uniform. These variations in the spectral features of the apatite crystals are indicative of some level of MC chemical diversity, and FTIR results were not sufficient to differentiate between benign and malignant TNs based on the characteristics of these MCs.

### Example 3

### The Morphology and Elemental Composition of FNA MCs

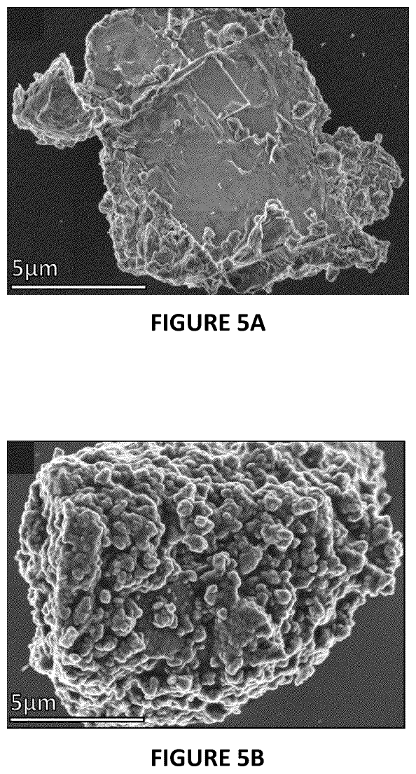

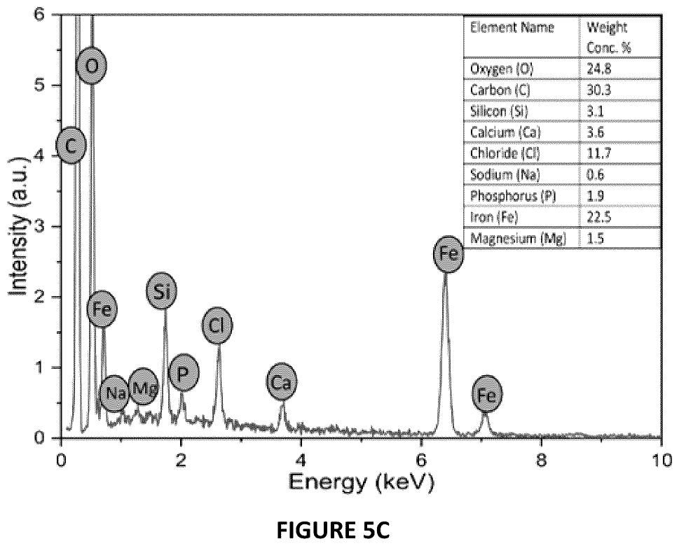

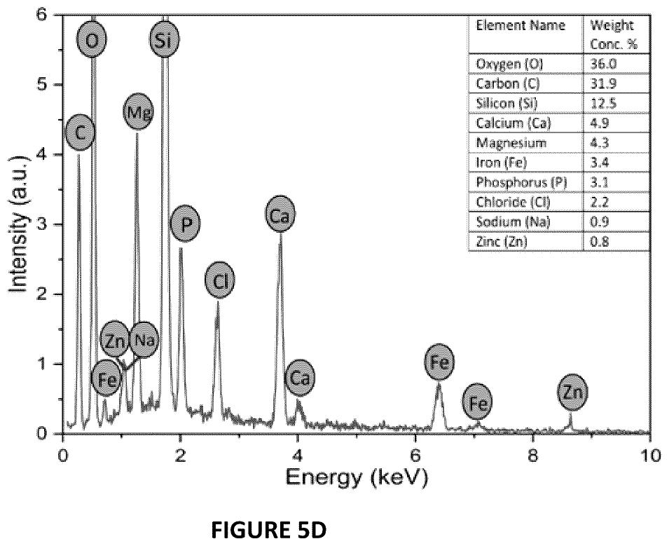

SEM was next used to explore whether FNA MCs isolated from benign and malignant TNs exhibited distinct morphological characteristics. While FTIR measurements indicated that FNA MCs obtained from both benign and malignant TNs were composed of carbonated apatite, they exhibited distinct morphological characteristics. While benign MCs were dense particles with clear edges 1-10 μm in size, malignant MCs were aggregates of sub-micrometer spherical particles tightly packed together with dimensions of tens of micrometers (FIGS. 5A-5B). Backscattered electron images show that the spherical particles produce a positive signal, implying that they are composed of a material with high electron density and not an organic material.

SEM-EDS was next used to investigate whether the benign and malignant FNA MCs differ not only in morphology but also in their elemental composition. Based on the EDS elemental composition, the precipitates were identified as MCs when they contained both calcium and phosphorous. Quantitative elemental analysis was not possible due to the rough surface texture of the MCs, and the EDS results were thus only used to determine the presence or absence of certain elements within these MCs. EDS characterization revealed that FNA MCs are chemically diverse and exhibit a heterogeneous elemental composition (FIGS. 5C-5D). In addition to Ca and P atoms, which are expected to be present in calcium phosphate apatite crystals, other elements were also detected including Mg, Zn, Fe, Na, Al, K, and Cl. The diversity in the elemental composition of the MCs was consistent with the chemical variations observed in the FTIR analyses. All MCs examined contained Ca, P and Fe, while the remaining detected elements were not present in all MC samples. Individual MCs detected in each sample also exhibited differences in their elemental composition. For example, seven different MCs were analyzed from one patient ultimately diagnosed with a benign TN, and while all seven of these MCs contained Cl, only three contained Mg, and none contained Zn.

Interestingly, FNA MCs obtained from benign TNs versus TC and neoplastic TN differed in their elemental composition (Table 2). A higher percentage of patients with TC and neoplastic TN had Mg in their FNA MCs as compared to patients with benign TNs (86% vs. 30%, respectively, p<0.001). Moreover, 91% of patients with a final diagnosis of TC and neoplastic TN had Zn in their FNA MCs, whereas MC samples from only 2/30 patients (7%) ultimately diagnosed with benign TNs contained Zn (p<0.001). Regarding the presence of Zn as a discriminator between benign and malignant TNs, all patients with indeterminate cytology and final diagnosis of TC or neoplastic TN (11/22) had Zn in their MCs. The two patients with false negative results had a cytological diagnosis of Bethesda 5 and 6. Two patients had false positive results: one with Bethesda 1 and one with Bethesda 3 cytological diagnosis, both with histopathology reporting adenomatoid (benign) nodule. All other 28 patients with a final diagnosis of a benign TN, 1 with Bethesda 1, 4 with Bethesda 3, and 23 with Bethesda 2 cytological diagnosis, did not have Zn in their MCs.

TABLE 2
Elemental profile of MCs in the material aspirated
from TNs grouped according to the final diagnosis

| | | Benign TN | TC or neoplastic TN * | |
|---|---|---|---|---|
| | | N = 30 | N = 22 | P-value |
| Percentage of | Ca | 100% | 100% | 1 |
| patients with | P | 100% | 100% | 1 |
| MCs containing | Mg | 30% | 86% | <0.001 |
| each element | Zn | 7% | 91% | <0.001 |
| out of the total | Fe | 100% | 100% | 1 |
| number of | Na | 56% | 82% | 0.011 |
| patients in each | Al | 40% | 59% | 0.17 |
| group | K | 50% | 50% | 1 |
| | Cl | 77% | 86% | 0.38 |

* Papillary thyroid cancer (PTC)- 17 patients, micro-PTC- 3 patients, Non-invasive follicular thyroid neoplasm with papillary-like nuclear features (NIFTP)- 1 patient, minimally invasive follicular thyroid cancer- 1 patient
Abbreviations:
Thyroid Nodule (TN),
Thyroid cancer (TC),
Microscopic calcifications (MCs)

The levels of Zn detected in these FNA MCs by EDS ranged from 0.8-1.5 (w/w %). As these concentrations are close to the detection limit of the EDS measurement, the presence of Zn in the MCs was verified using Inductively Coupled Plasma-Optical Emission Spectrometry (ICP-OES), which is more sensitive to low Zn concentrations (Table 3). Using ICP-OES the inventors measured the Zn content in a hydroxyapatite standard with no added Zn as a Zn-free reference sample. Zn levels below the concentration detected in hydroxyapatite samples by ICP-OES were considered undetectable. ICP-OES results indicated that FNA MC samples from TC patients in which Zn was detected in EDS analyses also contained Zn concentrations above the levels in the hydroxyapatite standard, up to 0.5 wt %. FNA MCs from patients with benign TNs in which Zn was not detected via EDS, in contrast, contained Zn concentrations below those in the hydroxyapatite standard, less than 0.1 wt %.

TABLE 3
Comparison of the detection of
Zn in MCs using ICP-OES and EDS

| | Zn amount (ppb) | Zn |
|---|---|---|
| | detected by | presence by |
| | ICP-OES | SEM-EDS |
| Standard-Hydroxyapatite | 1.6 | No |
| Patients with benign TNs (range) | 0.5-1.4 | No |
| Patients with malignant TNs (range) | 7.3-9.7 | Yes |

Based on a combination of these SEM-EDS, ICP-OES, and FTIR results, it is apparent that the FNA MCs isolated from patients TNs consist of carbonated calcium orthophosphate in the form of apatite containing several elements in addition to Ca and P. MCs isolated from benign and malignant TNs exhibit distinct morphological characteristics and elemental profiles. While almost all malignant MCs contain Zn, most benign MCs do not.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. (canceled)

2. A method for diagnosing and treating a thyroid cancer in a subject in need thereof, the method comprising:
a. determining presence, amount, or both, of at least one species of Zinc, Magnesium, or both, wherein said species is in a MC derived from a thyroid biopsy obtained from said subject, wherein: (a) the presence of said species; (b) alteration in the amount of said species compared to a healthy control; or (c) both, in said MC is indicative of said subject being afflicted with said thyroid cancer, thereby diagnosing thyroid cancer in the subject; and
b. administering to said subject diagnosed with thyroid cancer of step (a) a therapeutically effective amount of an anti-thyroid cancer therapy.

3. The method of claim 1 or 2, wherein said at least one species comprises Zinc, and presence of said Zinc in said MC is indicative of the presence of at least one malignant thyroid nodule in said thyroid biopsy.

4. The method of claim 3, wherein absence of said Zinc in said MC is indicative of the presence of a benign thyroid nodule in said thyroid biopsy, absence of a malignant thyroid nodule in said thyroid biopsy, or both.

5. The method of claim 2, wherein said thyroid cancer is differentiated thyroid carcinoma.

6. The method of claim 5, wherein said differentiated thyroid carcinoma is papillary thyroid carcinoma.

7. The method of claim 2, wherein said healthy control comprises at least one benign thyroid nodule and is devoid of a malignant thyroid nodule.

8. The method of claim 2, further comprising a step before said determining, comprising obtaining a thyroid biopsy from said subject.

9. The method of claim 8, wherein said obtaining is by fine-needle aspiration (FNA).

10. The method of claim 8, further comprising a step after said obtaining and before said determining, comprising preparing said sample for X-ray based analysis.

11. The method of claim 10, wherein said X-ray based analysis comprises energy dispersive X-ray spectroscopy (EDS).

12. The method of claim 8, further comprising a step after said obtaining and before said determining, comprising preparing said sample for inductively coupled plasma (ICP).

13. The method of claim 12, wherein a determined wt % percentage of at least 0.09 wt % of said Zinc in said MC is indicative of the presence of at least one malignant thyroid nodule in said thyroid biopsy.

14. The method of claim 13, wherein said determined is by ICP.

15. A method for treating thyroid cancer in a subject diagnosed therewith, the method comprising administering to said subject a therapeutically effective amount of an anti-thyroid cancer therapy, wherein said diagnosed is by determination of: (a) presence of at least one species selected from: Zn, Mg, or both; (b) alteration of amount of said species compared to a healthy control; or (c) both (a) and (b), in a sample comprising a thyroid biopsy being obtained or derived from said subject, wherein said species is in a MC.

16. A method for preparing a sample comprising a thyroid biopsy for thyroid cancer diagnosis, the method comprising providing a sample comprising a thyroid biopsy and extracting a MC comprising at least one species selected from: Zn, Mg, or both, from said thyroid biopsy, thereby preparing the sample comprising a thyroid biopsy for cancer diagnosis.

17. The method of claim 16, wherein said extracting comprises subjecting said sample to a fixation solution, eliminating or removing an organic material from the fixed sample, or both.

18. The method of claim 17, wherein said eliminating or removing said organic material comprises subjecting said fixed sample to a basic solution.

19. The method of claim 18, wherein said basic solution comprises sodium hypochlorite.

20. The method of claim 16, wherein said thyroid biopsy is obtained by FNA.

21. The method of claim 16, wherein said prepared sample is suitable for determination of presence, amount, or both, of said at least one species selected from: Zn, Mg, or both, and being in a MC, by an X-ray based methodology, optionally wherein said X-ray based methodology comprises EDS.

22. (canceled)
